(19) United States
(12) Patent Application Publication
(10) Pub. No.: US 2016/0367719 A1
(43) Pub. Date: Dec. 22, 2016

(54) FRACTIONS OF EXTRACTS OF HELICHRYSUM HAVING MUCOHADHESIVE PROPERTIES

(71) Applicant: ABOCA S.P.A SOCIETÀ AGRICOLA, Sansepolcro (IT)

(72) Inventors: Valentino MERCATI, Sansepolcro (IT); Luisa MATTOLI, Sansepolcro (IT)

(21) Appl. No.: 15/121,248

(22) PCT Filed: Feb. 11, 2015
(86) PCT No.: PCT/IB2015/051018

§ 371 Date: Aug. 24, 2016

(30) Foreign Application Priority Data
Feb. 24, 2014 (IT) RM2014A000081

(51) Int. Cl. A61L 26/00; A61L 15/58; A61L 15/40; A61K 36/28

(52) U.S. Cl. CPC A61L 26/0057; A61K 36/28; A61L 26/0061; A61L 15/58; A61L 15/40; A61K 2236/33; A61K 2236/55

(57) ABSTRACT

The present invention relates to fractions of extracts of Helichrysum having mucoadhesive and barrier effect properties, compositions comprising said fractions, their uses and methods for the preparation of said fractions and said compositions.

The present invention relates to fractions of extracts of Helichrysum depleted in active principles having mucoadhesive properties, and having barrier effect, compositions comprising said fractions, their uses and methods for the preparation of said fractions and said compositions.

### PRIOR ART

Helichrysum (syn. Helychrisum italicum) is a perennial plant belonging to the genus of the Asters, with a shrubby habit, high 30-40 cm, of gray-whitish color with alternate, linear leaves, 20-40 mm long and 1 mm wide, covered by a fine whitish down; soft and convoluted.

The inflorescence is a corymb comprised of numerous flowering tops, placed at the stalk apex, with tubulous flowers of a golden-yellow color.

From a chemical standpoint the active principles of Helichrysum are mainly represented by flavonoids, and methodologies for obtaining Helichrysum extracts particularly rich in active principles have been described in the literature, e.g. in Patent EP 1883415 to the same Applicant.

As is well-known, Helichrysum is used for its emollient properties, due, as mentioned above, mainly to the flavonoids present above all in the lipophilic part extracted from the flowering tops of the plant.

### SUMMARY OF THE INVENTION

The authors of the present invention tried to exploit also the non-lipophilic part of the extract of Helichrysum and discovered, by analyzing the properties of the hydroalcoholic extract of Helichrysum, that said extract does not seem to possess substantial mucoadhesive properties, while it has some degree of barrier effect. This means that in general the extract of Helichrysum tops does not have particularly detectable mechanical (mucoadhesion and barrier) activities.

The authors of the present invention then tried to understand whether it would be possible to isolate fractions of extract of Helichrysum possessing better mechanical activities and less pharmacological activities, so as to be able to use commonly discarded components of this plant that would have interesting, mainly mechanical activities.

The authors of the present invention therefore isolated various fractions of extract of Helichrysum and selected those fractions depleted in pharmacologically active principles (flavonoids) relative to the starting extract, which proved enriched in the mechanical properties of mucoadhesion and barrier effect.

By analyzing various fractions of hydroalcoholic extract of Helichrysum, it was surprisingly found that some fractions exhibit a strong mucoadhesive effect, an effect absent in the starting extract. Moreover, it was found that among these selected fractions some exhibit a barrier effect even greater than that of the initial extract.

In fact, comparative data obtained by the authors and reported in the detailed section below show that some fractions of hydroalcoholic extract of Helichrysum (only fractions selected in comparison with the starting extract are reported) have a mucoadhesion percentage much superior to that of the starting extract (in which mucoadhesion was equal to zero in two different assays used) and that some of these fractions also have a barrier effect greater than or equal to that measured in the starting extract.

The present invention therefore relates to fractions of extract of Helichrysum, characterized by having a mucoadhesion activity superior to that of said extract (which proved not measurable with two different measuring methods used) and a barrier effect greater than or comparable to that of said extract, compositions comprising said fractions, use of such fractions and of such compositions and methods for the preparation of such fractions.

Therefore, object of the invention are fractions of extract of Helichrysum, characterized by having a mucoadhesion activity superior to that of said extract and a barrier effect greater than or similar/equal to that of said extract, compositions comprising said fractions, use of such fractions and of such compositions and methods for the preparation of such fractions.

Further object of the invention are compositions comprising one or more fractions of extract of Helichrysum as described herein and defined in the claims and not comprising non-fractioned extract of Helichrysum and/or fractions different from the ones described herein.

Object of the invention is also a carrier for pharmaceutical or herbal compositions comprising one or more fractions of extract of Helichrysum as described herein and as defined in the claims, characterized by having a mucoadhesion percentage in mucoadhesion assay on cells greater than 60%.

In this case as well, the carrier does not comprise non-fractioned extract of Helichrysum and/or fractions different from those described herein.

Finally, object of the invention is a process for the preparation of fractions of extract of Helichrysum having mucoadhesive properties and barrier effect, characterized by the following steps:

a. preparing a hydroalcoholic extract of Helichrysum and subjecting said extract to centrifugation and/or decantation, thereby obtaining a precipitated fraction and a clarified alcoholic fraction;

b. obtaining from the clarified alcoholic fraction prepared in a. a concentrated aqueous fraction B

wherein said fraction B is characterized by a mucoadhesion activity superior to that of said extract and by a barrier effect greater than or equal to the barrier effect of said extract.

### GLOSSARY

By “EXTRACT OF HELICHRYSUM” to the ends of the present invention it is meant a hydroalcoholic extract of Helichrysum tops.

NMWCO=Nominal Molecular Weight Cut-off

PERMEATE: extract that has passed through the semi-permeable ultrafiltration or nanofiltration technique

RETENTATE: extract that did not pass through the ultrafiltration or nanofiltration membrane or material adsorbed on resin.

ELUATE: material not adsorbed on resin following adsorption passages thereon.

ULTRAFILTRATION: filtration technique on semi-permeable membrane characterized by pores having sizes of 10,000 daltons (0.01 μm) to 500 daltons (about 0.005 μm).

NANOFILTRATION: filtration technique on semi-permeable membrane characterized by pores having sizes of 500 to 150 daltons.

TOPS or FLOWERING TOPS By “tops”, or “flowering tops” it is meant the term as commonly used in herbal medicine and in botanical treatises, therefore the aerial ends of the plant which contain leaves, stems (meant as branches and not as main stalk of the plant) and flowers.

By “mucoadhesion activity” it is meant in the present invention the ability of a compound or of a substance to adhere to the mucosal surface.

By “barrier effect” (BE) it is meant the ability of a compound or of a substance to create a protective film on a cell surface, such as, e.g., the mucous membrane or the skin.

By “mucoadhesion assay on cells” it is meant in the present description the assay for measuring the mucoadhesive activity as described in the detailed section and in the examples section.

### DETAILED DESCRIPTION OF THE FIGURES

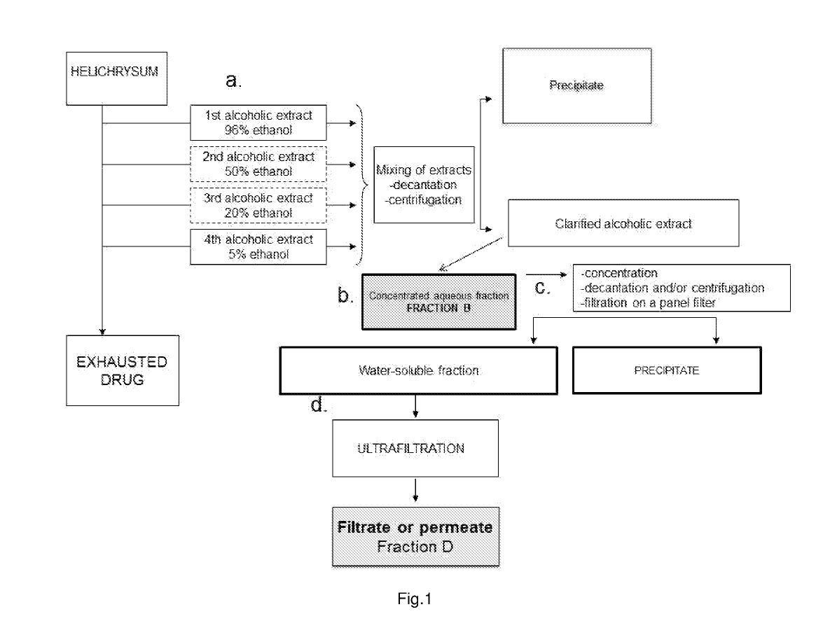

FIG. 1 is a block diagram depicting the processes for the preparation of the fractions of the invention

### DETAILED DESCRIPTION OF THE INVENTION

As described in the above summary, the present description relates to fractions of extract of Helichrysum having mechanical features, such as mucoadhesive ability and barrier effect, improved with respect to the non-fractioned starting extract.

The authors of the present invention have in fact examined the mucoadhesive and barrier effect abilities of the whole (non-fractioned) extract of Helichrysum, such as, e.g., the hydroalcoholic extract of Helichrysum flowering tops, and have found that such extract did not have a mucoadhesive ability measurable in standard experiments for mucoadhesion measurement, like the mucoadhesion assay on an inclined plane or the mucoadhesion assay on cells, whereas it had a net barrier effect present and measurable, e.g., by a simple assay reported in the experimental section of this description.

By fractioning with various techniques the extract of Helichrysum and analyzing the obtained fractions, the authors of the present invention have surprisingly isolated some fractions that exhibit mucoadhesive abilities much greater than the starting extract and a barrier effect greater than or equal with respect to the barrier effect of the starting extract.

In a first embodiment, the present invention relates to fractions of extract of Helichrysum characterized by a mucoadhesion ability or activity superior to that of the starting extract, i.e. superior to those of the extract from which said fractions are separated, and by a barrier effect greater than or equal with respect to that of the starting extract.

In the present invention, the mucoadhesion activity could be measured according to any method known to the technician in the field. In particular, it could be measured as mucoadhesion percentage by a simple cytoadhesion assay (also defined herein as mucoadhesion assay on cells) performed with the fractions of the invention, which aims at assessing the mucoadhesivity of the product under examination in order to establish whether such product has the ability to adhere to mucous membranes and consequently exert a protective action.

This mucoadhesive activity can, e.g., be measured on mucosal cells, by evaluation of the percentage of inhibition of the lectin-glycoprotein bond induced by pretreatment of the analyzed cells with one or both of the fractions described and claimed in the present application.

In short, mucoadhesivity is determined by evaluation of the percentage of inhibition of the lectin-glycoprotein bond induced by the analyzed sample using buccal, gastric, intestinal or vaginal mucosal cells pretreated with the sample under examination. Cells are initially pretreated for a variable time (e.g., 15-30 minutes) with the sample under examination, and are subsequently treated with biotinylated lectin (Con-A), to which streptavidin peroxidase is subsequently added, making it possible to form the protein-glucose-lectin-biotin-streptavidin peroxidase complex. At this point, the cells are washed and the protein-glucose-lectin-biotin-streptavidin peroxidase complex is quantified, thanks to the presence of the peroxidase, by means of a reaction of oxidation of the ortho-phenylenediamine.

The protein/glucose/lectin/biotin/streptavidin peroxidase complex catalyses the polymerization reaction:

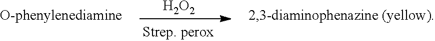

The intensity of the yellow/orange coloration of the solution (measured using a spectrophotometer with =450 nm) is proportional to the quantity of glycoprotein/lectin bonds and therefore to the quantity of available sites (glycoproteins) for mucoadhesion. The absorbance value thus determined constitutes the “control”.

In the mucoadhesion assay on cells reported below, the mucosal cells are treated for about 15 minutes at 30° C. with the product under examination before the treatment with lectin. In the presence of mucoadhesive products, said products will inhibit lectin bonding, decreasing, proportionally to their mucoadhesion ability, the signal strength in the sample compared to the control as described above.

The percentage of mucoadhesion of the product (% MA) is determined as % MA=(1−abs sample/abs control)×100

In a particular embodiment, the mucoadhesion percentage of the fraction of the invention is greater than 60%, or greater than 65% or greater than 70% in mucoadhesion assays on cells as those described above and described in detail in the examples.

It is of interest to note that the starting extract has a mucoadhesion percentage, calculated with the same assay, equal to zero.

The fractions of extract of Helichrysum described herein exhibit also a barrier effect greater than or equal to that of the starting extract.

The authors of the present invention have in fact found that some fractions among those selected on the basis of the desired mucoadhesive abilities also exhibited a barrier effect greater than or equal to that of the starting extract.

The barrier effect of a compound on mucous membranes or skin may be measured in any way available to the technician in the field. For instance, the barrier effect may be measured by cellular markers release inhibition assays in response to an inflammatory or irritating agent as described in detail in the experimental section below.

In short, however, an in vitro method for the assessment of the barrier effect of fractions of extract of Helichrysum on a cell culture can be summarized with the following steps:

a) preparing a cell culture;

b) placing the sample of interest (e.g. a fraction of Helichrysum extract) on a semi-permeable membrane separating it from said culture, and, subsequently or concomitantly, adding to said sample a substance (e.g., an inflammatory agent such as bacterial lipopolysaccharide LPS or an interferon; an irritating or allergenic agent like ovoalbumin, dinitrochlorobenzene, oxazolone, eugenol, penicillin G, nickel sulphate) that, when in contact with said culture, be able to induce release of a marker (e.g., histamine or -glucuronidase or a cytokine like IL-8, IL-6, IL1, TNF, TNF) from said culture;

c) detecting in one or more successive time instants the concentration of said marker in the experiment prepared in a) and b) and comparing the measurements obtained relative to one or more reference values, wherein the lack of increase over time of said marker in step c. denotes a barrier effect of the sample (or even an increase of marker measurable only over a longer time with respect to the control, in which the sample under examination is not placed on the semipermeable membrane.

In particular, step c) can comprise:

detecting in one or more successive time instants the concentration of said marker in the experiments prepared in a) and b) and in a parallel control experiment in which the Helichrysum extract fraction is not added on the permeable membrane in

b) and comparing the measurements obtained from the two experiments in the same time instants.

Step c) could also comprise:

detecting in one or more successive time instants the concentration of said marker in the experiment prepared in steps a) and b) relative to the amount of said marker measured in the same experiment before step b).

In one embodiment the method can further comprise a further step of preparing an internal control in which cultured cells are pre-treated with the substance adapted to induce marker release and the fraction of an extract of Helichrysum is placed on the semi-permeable membrane in the absence of said substance, which could be added later to the fraction under examination in case said substance were to settle before said fraction.

According to one embodiment, the barrier effect (BE) is measured by IL-6 inhibition assay in response to an agent such as LPS.

BE=% reduction in release of cytokine IL-6=100−[(pg/μL cytokines released from sample/pg/μL cytokines released from C+)×100]

In an interesting embodiment, the Helichrysum extract fraction of the invention has a barrier effect percentage in cellular markers release inhibition assays in response to an inflammatory or irritating agent (like, e.g., inhibition of cytokines, such as IL-6 and/or IL-8, release as in response to LPS) greater than or equal to 30%, or even greater than or equal to 32% like, e.g. a barrier effect percentage of about 34% or of about 49%.

In a particular embodiment, the fraction of extract of Helichrysum according to the invention is characterized by a mucoadhesion activity superior to that of said starting extract and a barrier effect percentage in cellular markers release inhibition assays in response to an inflammatory or irritating agent (like, e.g., cytokines release inhibition, e.g. IL-6 and/or IL-8 in response to LPS) of about 49%. The above-indicated fraction, when mucoadhesion is measured by mucoadhesion assay on cells, has a mucoadhesion percentage comprised between 65 and 70%, i.e. a mucoadhesion percentage greater than 65%, e.g. of about 68%. In another embodiment, the fraction of extract of Helichrysum according to the invention is characterized by a mucoadhesion activity superior to that of said starting extract and a barrier effect percentage in cellular markers release inhibition assays in response to an inflammatory or irritating agent (like, e.g., cytokines release inhibition, e.g., IL-6 and/or IL-8 in response to LPS) greater than 30%, e.g. of about 34%.

The above-indicated fraction, when mucoadhesion is measured by mucoadhesion assay on cells, has a mucoadhesion percentage greater than 70%, e.g. a mucoadhesion percentage of about 74%.

In a preferred embodiment of the invention, the starting extract of Helichrysum, from which the fractions of the invention are separated, is a hydroalcoholic extract of Helichrysum flowering tops as defined above, which could optionally be dried or lyophilized.

The fractions could optionally be then dried or lyophilized.

Object of the invention are also compositions comprising fractions of extract of the invention or mixtures thereof. The compositions according to the invention do not comprise non-fractioned extract of Helichrysum, like, e.g., non-fractioned hydroalcoholic extract of Helichrysum and/or fractions of extract of Helichrysum different from those described herein. The sole compounds coming from Helichrysum in the compositions are therefore those present in the fractions as described and claimed herein.

The fractions of the invention, their mixtures, or compositions comprising such fractions or mixtures as described above can be used as mucoadhesive carriers for pharmaceutical or herbal compositions.

The fractions of the invention therefore provide a new source of material of plant origin useful for the protection and/or the repair of the mucous membranes and of the skin, and/or also as excipient/carrier capable of retaining for a longer time desired substances on the mucous membranes, wherein the pharmacological effects attributable to the components known as active principles are substantially reduced or even eliminated.

Said condition is particularly desirable when materials of natural or plant origin are to be used for actions of a mainly mechanical type (i.e., not for pharmacological actions having, for instance, a direct effect on the activation of receptorial, immune pathways or metabolic processes). The use of substances having a mainly mechanical effect, such as, e.g., substances having a barrier or mucoadhesive effect, is desirable, for instance, to cooperate with a pharmacological therapy. By minimizing the presence of substances with pharmacological activity (i.e., flavonoids) in the material of plant or natural origin as in present invention, it is obtained a material with a mainly mechanical effect that can be used in combination with drugs, minimizing possible undesired interactions with drugs, and at the same time exploiting the typical feature of substances of plant origin, of having, besides the pharmacologically active components commonly used, which in this case are substantially eliminated, a vast number of components having a mechanical effect, capable of exerting a protective effect on the skin and mucous membranes.

By “protection of the mucous membranes” it is meant a mechanical protection, given by the mucoadhesive or barrier abilities of the fractions of the invention or of mixtures thereof, which could be associated with a pharmacological protection related to active principles different from those of Helichrysum, mixed with the fractions of the invention. Just as a bandage physically protects a wound and prevents further worsenings of the same, thereby facilitating its healing, the barrier effect of the fractions of the invention protects the skin and mucous membranes from aggression of principles that may cause pathologies and irritations and that can slow down the healing of compromised skin or mucous membranes. In the present description said mucous membranes can be the oral mucosa, gastric mucosa, intestinal mucosa, the nasal mucosa, the vaginal mucosa, the uterine mucosa, the rectal mucosa.

The fractions of the invention could be used alone or mixed with each other and could be optionally further mixed (alone or in combination) with extracts of other plants and/or natural active principles of other origin (i.e., not extracted from Helichrysum) as active principles of other plants or of other natural products in order to boost the mechanical activity, as defined above, of the resulting mixtures. Possible formulations containing the fractions of extract according to the invention will subsequently be described in more detail.

There will presently be provided processes enabling a person skilled in the art to isolate fractions of extract of Helichrysum meeting the above-indicated requirements.

Evidently, starting from these indications the technician in the field could develop other processes enabling to obtain fractions meeting the above-indicated criteria with no need of additional inventive activity. In fact, once known that fractions of extract of Helichrysum exhibiting peculiar mucoadhesive and barrier effect properties do exist, and once known the processes enabling to isolate them, the technician in the field could effect modifications to these processes and obtain analogous fractions without particular use of inventive activity.

Hence, the processes described herein are to be understood as teachings that in no way limit the invention, rather as implementation examples to the benefit of the technician in the field.

The present invention therefore relates also to a process for the preparation of fractions of extract of Helichrysum characterized by the following steps:

a. preparing a hydroalcoholic extract of Helichrysum and subjecting said extract to centrifugation and/or decantation, thereby obtaining a precipitated fraction and a clarified alcoholic fraction;

b. obtaining from the clarified alcoholic fraction prepared in a. a concentrated aqueous fraction (polar or water-soluble fraction) referred to as fraction B wherein said fraction B is characterized by a mucoadhesion percentage greater than the mucoadhesion percentage of said extract and a barrier effect greater than or equal to the barrier effect of said extract.

In particular, said fraction B is characterized by a mucoadhesion percentage measured with mucoadhesion assay on cells greater than 60% (greater than 65%, of about 68%) and a barrier effect in cellular markers release inhibition assays in response to an inflammatory or irritating agent (like, e.g., cytokines release inhibition, e.g. IL-6 and/or IL-8 in response to LPS) greater than 45% (of about 49%)

According to a further embodiment, the process of the invention can also comprise the following steps:

c. decanting and/or centrifuging and filtering said fraction B, thereby collecting a water-soluble fraction;

d. subjecting the water-soluble fraction obtained in c. to membrane ultrafiltration or nanofiltration and collecting a fraction D, corresponding to the permeate obtained from said ultrafiltration or nanofiltration, wherein said fraction D is characterized by a mucoadhesion activity superior to that of said extract and a barrier effect comparable to that of said extract.

In particular, said fraction D is characterized by a mucoadhesion percentage, in an assay on cells, greater than 70% and a barrier effect greater than or equal to 30%, or greater than or equal to 32%.

The hydroalcoholic extract prepared at a. is, in an embodiment of the invention, an alcoholic extract of Helichrysum flowering tops.

The properties of the fractions obtained with the above-described processes are summed up in the following Table 1:

| | NET BARRIER | | |
|---|---|---|---|
| | EFFECT: % IL-6 | | |
| | inhibition | | Total |
| SAMPLE | of the B.A. -% | Mucoadhe- | flavonoids |
| (Extract or | IL-6 inhibition | sion on | as isoquercitrin |
| Fraction) | of the CI | cells % | SEM(%) |
| HELICHRYSUM | 32 | 0 | 0.82 |
| HYDROALCOHOLIC | | | |
| EXTRACT, DRY | | | |
| HELICHRYSUM, | 49 | 68 | 0.33 |
| FRACTION B | | | |
| HELICHRYSUM, | 34 | 74 | 0.19 |
| FRACTION D | | | |

Let us remind that the BE of a substance or compound is expressed herein as % reduction in the release of IL-6 cytokine by cells exposed to LPS wherein the sample was assayed (TB) with respect to the positive control (C+ or C−) wherein the cells have only been exposed to LPS

The initial fraction of extract of Helichrysum has a percentage of total flavonoids titrated as isoquercitrin equal to about 0.82%, whereas fraction B of the extract has a percentage of total flavonoids titrated as isoquercitrin equal to about 0.3% and fraction D has a percentage of total flavonoids titrated as isoquercitrin equal to about 0.2%.

The process described herein could be carried out starting from Helichrysum tops, both fresh and dried, or alternatively starting directly from fluid (like, e.g., hydroalcoholic extracts) or dry Helichrysum extracts.

The dry extracts could be resuspended/resolubilised by means of hydroalcoholic solutions, e.g. by using a solid/solvent ratio of 1:5, or 1:7 or 1:8 or 1:10, putting under stirring the mass for a time ranging from 4 to 8 hours. Step a. of the process:

a. preparing or using a hydroalcoholic extract of Helichrysum tops and subjecting said extract to centrifugation and/or decantation, obtaining a clarified alcoholic extract;

According to the invention, the above-described process can be carried out by performing at step a. at least two, at least three, or at least four steps in ethanol at decreasing concentrations, wherein the decreasing concentrations of ethanol are from about 96% ethanol to about 0% ethanol.

For instance, step a. of the process can be carried out by performing a step in about 96% ethanol, and a step in about 5% ethanol.

Or, step a. of the process could be carried out by performing a step in about 96% ethanol, a step in about 50% ethanol and a step in about 20% ethanol.

Or, step a. of the process could be carried out by performing a step in about 96% ethanol, a step in about 50% ethanol and/or a step in about 20% ethanol and a step in about 5% ethanol.

The extracts obtained as described above are gathered to form a hydroalcoholic extract. These can be gathered, e.g., in equal mutual ratios, such as 50:50 in case of two steps in ethanol, or such as 33.3:33.3:33.3 in case of three steps in ethanol, or 25:25:25:25 in case of four steps in ethanol. Evidently, the extracts obtained by performing the various steps in ethanol described above could be gathered into different ratios according to the general knowledge of the technician in the field.

As already indicated above, the clarified alcoholic fraction obtained at step a. is used in the next step b. to prepare a concentrated aqueous fraction (fraction B).

Such concentrated aqueous fraction, or fraction B, could be prepared by concentrating the hydroalcoholic fraction obtained in a. by alcohol evaporation, then it is decanted and/or centrifuged and/or filtered and the resulting concentrated aqueous fraction is collected. For instance, in case of decantation and/or centrifugation, the aqueous supernatant is collected whereas in case of filtration the filtrate is collected. Alternatively, alcohol evaporation could be performed after the centrifugation and/or decantation and/or filtration steps.

Decantation can be performed, e.g., 1 to 72 hours, and can be followed or replaced by one or more centrifugations to about 3000-4000 (e.g., about 3500) rpm for a period of between 1 and 10 minutes, e.g. 5 minutes. For the filtration suitable filters known to a technician in the field can be used, like, e.g., panel filters having a cutoff of about 25 micrometers. In some cases, further consecutive filtrations of the extract can be performed on filters having gradually decreasing cutoffs, from 200 micrometers down to 0.1 micrometers.

The supernatant or the filtrate obtained after this step or steps is then collected and subjected, in case this has not already been done before, to alcohol evaporation by standard techniques, like e.g. by use of a thin film distillation system, or a batch concentration system according to standard protocols commonly used by the technician in the field.

The result of this evaporation is a concentrated aqueous fraction, herein referred to as fraction B.

Fraction B thus obtained has a mucoadhesion ability superior to (greater than) that of the starting extract and a barrier effect greater than that of the starting extract. In particular, the mucoadhesion percentage of fraction B measured by mucoadhesion assay on cells is greater than 60% or even greater than 65%, for instance equal to about 68% and the barrier effect, measured by cellular markers release inhibition assays in response to an inflammatory or irritating agent, is greater than 45%, for instance equal to about 49%.

Fraction B can be further subjected to a further process of substances depletion by ultrafiltration or nanofiltration.

Fraction B can be subjected to decantation for a period of between 1 and 72 hours, and the supernatant is then collected, thereby obtaining a clarified or clear solution.

Decantation could be followed or replaced by one or more centrifugations to about 3000-4000 (e.g., about 3500) rpm for a period of between 1 and 10 minutes, e.g. about 5 minutes.

Alternatively, fraction B is filtered, thereby obtaining a clarified solution. In some cases, further consecutive filtrations of the extract can be performed on filters having gradually decreasing cutoffs, starting from 200 micrometers until arriving to 0.1 micrometers. The filtration can be performed, for instance, on a panel filter having a cutoff of 25 micrometers.

In a further embodiment fraction B could be decanted as described above and the supernatant obtained could then be filtered as described above.

The supernatant or the filtrate obtained as described above are recovered and subjected to a further step of depletion in pharmacologically active aromatic substances by ultrafiltration (or nanofiltration) or passage on adsorbing resin.

The method of the invention may therefore comprise a further step, described below as d, of depletion of undesired substances from the supernatant or the filtrate obtained in step c. described above before collection of the desired extract, wherein said supernatant or filtrate is subjected to one or more further steps of depletion in active principles.

This step could be carried out by ultrafiltration or nanofiltration of the supernatant or filtrate obtained in c. (step d.).

The supernatant or filtrate obtained in c. is subjected to one or more steps of ultrafiltration or nanofiltration and the filtrate is recovered as fraction D, characterized by a mucoadhesion activity superior to that of the starting extract and a barrier effect superior to that of the starting extract, i.e. of the extract at a. (see description of fraction D above).

In this further step, fraction B is subjected to one or more steps of ultrafiltration on membrane with a molecular weight cutoff (NMCWO) of from 500 to 15,000 daltons, e.g. of 10,000 daltons, 5,000 daltons, 2,500 daltons, 1,000 daltons, or of nanofiltration on membrane with a cutoff lower than 500 daltons. The filtrate thus obtained (fraction D) can be used as is or subjected to a lyophilization process to provide a lyophilized extract useful for formulations having therapeutic employ for internal and/or external use.

According to the invention, therefore, said one or more ultrafiltration steps could be performed on a membrane having a cutoff of about 500-15,000 daltons, e.g. about 10,000 daltons, 5,000 daltons, 2,500 daltons, 1,000 daltons, whereas the nanofiltration one could be performed on a membrane having a cutoff lower than 500 daltons.

The present invention therefore relates to a process for the preparation of fractions of extract of Helichrysum having a high mucoadhesive power, comprising the following steps:

a. preparing a hydroalcoholic extract of Helichrysum flowering tops and subjecting said extract to centrifugation and/or decantation, thereby obtaining a precipitated fraction and a clarified alcoholic fraction;

b. obtaining from the clarified alcoholic fraction prepared in a. a concentrated aqueous fraction referred to as fraction B.

c. decanting and/or centrifuging and filtering said fraction B, thereby collecting a water-soluble fraction;

d. subjecting the water-soluble fraction obtained in c. to membrane ultrafiltration or nanofiltration and collecting a fraction D, corresponding to the permeate obtained from said ultrafiltration or nanofiltration, wherein said fraction D is characterized by a mucoadhesion activity superior to that of said extract and a barrier effect comparable to that of said extract.

In particular, fraction D obtained at step d. is characterized by a mucoadhesion percentage, when measured by mucoadhesion assay on cells, greater than 70% and a barrier effect, when measured by cellular markers release inhibition assays in response to an inflammatory or irritating agent, greater than or equal to 30% o greater than or equal to 32%, e.g. equal to about 34%.

As already indicated above, the mucoadhesion ability of the initial extract of Helichrysum as described herein is not detectable by assay of mucoadhesion to cells; this means that when the Helichrysum extract is used as sample to be assayed in the assay described below, the results obtained with the sample equal those obtained with the “blank” or negative control, in which no substance is contacted with the cells before the lectin treatment.

The process according to the present invention therefore enables to obtain, fractions of extract of Helichrysum as described above, depleted in active principles, and surprisingly rich in substances having a protective action.

The present invention also relates to a composition comprising one or more fractions of extract of Helichrysum useful in the protection of the skin or of the mucous membranes, wherein said fractions are substantially depleted in active principles and exhibit mucoadhesive properties, and optionally also barrier effect ones, initially low or absent in the starting extract.

Such a protection, of course with reference both to the fractions per se or mixed with each other and to the composition, could be a preventive protection or a protection of partially compromised skin or mucous membranes as already described above. The compositions of the invention comprise solely the fractions of the invention as regards Helichrysum.

The composition or the fractions of extract can, by virtue of their barrier effect and mucoadhesive effect, facilitate the repair of damaged mucosal or skin tissue with entailed restoration of healthy and elastic skin or mucous membrane.

The skin lesions according to the present invention may be, in case no cicatrizing and/or antimicrobial active principles be associated to the fractions of the invention, lesions that may involve also tissue underlying the skin and in which no open wounds are present.

By skin lesions not implying the presence of open wounds, according to the present description, are meant those lesions in which the superficial layer of the skin, and the underlying layers, though not being wounded, are particularly fragile, irritated and damaged.

Non-limiting examples of this type of lesions are represented by first-degree burns, first-degree decubitus lesions, pressure lesions, newly cicatrized rashes, wounds or burns, irritations, erythemas. in association with active principles suitable for the treatment of skin lesions with open wounds, the fractions of the invention could be as adjuvants of such treatment thanks to their mucoadhesion effect or barrier effect.

The compositions or the fractions (or mixtures thereof) of the invention could then be used for the treatment or the prevention of skin lesions not implying the presence of open wounds, or in the prevention or slowing down of worsenings of the same or, in association with suitable active principles of plants different from Helichrysum, in the treatment of skin or mucosal lesions with open wounds.

The compositions according to the invention could advantageously comprise further components, e.g. of plant origin, having, e.g. emollient, digestive, pro-kinetic, cholagogic, carminative, prebiotic, relaxing, excipient, preserving, wetting properties, besides active principles of natural origin of plants different from Helichrysum.

When the composition or one or more of the fractions of the invention are used for the protection of the skin, the application thereof will be topical. The embodiments of the compositions for topical use are reported hereinafter in the description.

The composition according to the invention could be made, e.g., as oil-in-water emulsion, water-in-oil emulsion, oil-in-gel or gel-in-oil emulsion, multiple emulsions, sprays, and anhydrous formulations (pomade, gel, paste, cream, ointment) and will comprise one or more excipients suitable for the making of the desired end form.

Such excipients could be, e.g., emulsifying agents (cetearyl alcohol, cetearyl glucoside, hydrogenated castor oil) rheological additives, antioxidants (like, e.g., vitamins, tocopherols or other antioxidants known in the field).

The emulsifying agent could be a surfactant, which by lowering the interfacial tension decreases the free energy of the system; alternatively, also non-surfactant substances can be used, such as gum arabic, gelatin, hydrophilic colloids or finely subdivided powders (e.g., talc). In one embodiment, the excipients could be present at an overall concentration in weight comprised between 3 and 8%, such concentration being of course indicative, taking into account that anyhow the technician in the field will know how to adapt the concentration of the necessary excipients according to the embodiment he/she intends to prepare without addition of inventive activity.

Moreover, the composition could comprise perfuming and/or coloring agents which give it a pleasant smell, like, e.g., one or more essential oils like, e.g., lavender essential oil, melaleuca essential oil, lemon, mint, orange, and/or coloring agents which allow, e.g., to easily recognize the areas of application of the composition, the coloring being, e.g., temporary, so that it does not interfere with other later applications of the composition.

In one embodiment, said coloring and/or perfuming agents are comprised at an overall concentration in weight of from 0.001 to 3%.

By “overall concentration in weight” it is meant the concentration in weight in the composition of the sum of the various excipients, or the concentration in weight in the composition of the sum of the various perfuming and/or coloring agents present in the composition.

As to the use of the composition in the protection of the skin, the invention also relates to medical devices like, e.g., medicated patches, medicated gauzes, medicated bandages, medicated towels, medicated pads, medicated diapers, i.e. patches, gauzes, bandages, towels, pads or diapers which comprise, or be at least partially covered by, or be at least partially imbued with the described composition of the invention in the most appropriate form.

The gauzes could be made as greasy gauzes, and so the bandages and the patches, using the composition made in the form, e.g., of an ointment, paste or cream. The towels could be imbued with the composition in the form of an oil emulsion with water or gel, and the diapers or the absorbent pads could be made by inserting the composition in the relevant layers known in the art for the insertion of protective or anti-irritating compositions.

Such products, like, e.g., infant diapers, absorbent pads (commonly called “sanitary pads”) for women and adult incontinence pads, are commonly used, e.g., in infancy-, women- and geriatrics-related fields, and the technician in the field will know where to insert the composition described herein and which embodiment be the most suitable one without the need of particular teachings and only based on conventional techniques in the art.

Such devices will be applicable to the part to be treated and/or protected for a preventive purpose.

As already indicated above, the composition of the invention may be a composition for topical use which could be realized in the form of oil-in-water emulsion, water-in-oil emulsion, multiple emulsions, sprays, and anhydrous formulations (pomade, ointment, gel, paste, cream, spray) according to techniques commonly used in the field. The formulation indicated herein as “spray” could be an anhydrous formulation or even a formulation in emulsion, the form with an atomizer enabling also self-applications in zones harder to reach (like, e.g., the back), useful in all those cases in which the formulation is used, e.g., to alleviate sun rashes, erythemas, or skin irritations of various kind.

According to another embodiment, one or more of the fractions of the invention or the composition of the invention could be used, thanks to their mucoadhesive and barrier effects, for the protection of the mucous membranes.

In this case as well, said protection could be a preventive protection, e.g. in all those cases envisaging an administration of drugs having the side effect of attacking the mucous membranes, or in those patients exhibiting conditions of recurring irritation of the same. In other cases, the protection could instead be a protection for curative purposes or in order to avoid the worsening of irritated or partially compromised mucous membranes.

In these cases, the administration of one or more of the fractions B or C of the composition could be topical for all those mucous membranes on which a topical administration is possible (e.g., oral (buccal), rectal, vaginal, nasal mucosa) or oral in the cases in which said membranes be, e.g., intestinal or gastric mucosal membranes.

The compositions for the protection of the mucous membranes could therefore be made in the form of capsule, tablet, lozenge, granule, powder, syrup, elixir, hard gelatine, soft gelatine, suspension, emulsion, solution, suppository, cream, gel, spray, ointment, pomade, paste, oil-in-water emulsion, water-in-oil emulsion, oil-in-gel emulsion, gel-in-oil emulsion.

In case of formulations for topical use, the above indications related to the compositions for the protection of the skin could be followed, or syrups, rinses, sprays could be made, e.g., for the protection of the mucous membranes of the mouth, throat, nose.

For application on the rectal mucosa, suppositories or enemas or microenemas could be used, with the excipients known to the technician in the field for the making of such compositions.

As already mentioned, the composition of the invention could be in the form of capsule, tablet, lozenge, hard gelatine, soft gelatine, granule, powder, syrup, elixir, suspension, emulsion. For oral administration the composition could be made in the form of daily unit dosages or of fractions of daily unit dosages (e.g. 2, 3, 4, 5, 6, or more capsules, tablets, lozenges, granule or powder single-doses, or gelatins could be taken over the day, in accordance with the judgment of the treating doctor), and may contain conventional excipients including, e.g., binding agents, like gum arabic, gelatine, sorbitol, gum tragacanth, and/or polyvinylpyrrolidone; fillers, like lactose, sugar, corn starch, rice starch, calcium phosphate, sorbitol and/or glycine; tableting lubricants, like magnesium stearate, talc, polyethylenglycol and/or silica; disintegrants, like potato starch; and wetting agents like sodium laurylsulphate. The tablets can be coated according to methods well-known in the standard pharmaceutical practice.

The composition could also be made in a liquid or semi-liquid form, as a suspension, emulsion, solution for oral administration, and could optionally contain natural aromatizing agents giving a palatable taste thereto.

The composition in the form of powder or granule could be pre-metered in suitable containers and ready for use, either by ingestion as such or to be resuspended in an appropriate liquid such as water, tea, etc. In this case as well, the composition could contain natural aromatizing agents giving a palatable taste thereto.

Evidently, all of the above-indicated excipients could be used in a pharmaceutically acceptable grade.

In one embodiment, the composition as described herein, in any one of the above-indicated embodiments, could be in the form of pharmaceutical composition, i.e. comprise pharmaceutical-grade ingredients, or it could be or be introduced into a special-purpose food (medical food) or into a medical device.

The composition according to the present description could be made in the form of pharmaceutical composition or of medical device according to any one of the classes described in Directive 93/42/EEC on medical devices (comprising also substances and not only “devices” in the mechanical sense of the term), or in the form of medical food, food supplement, or in any form according to the regulatory provisions of the Country in which said composition will be produced.

The medical device or the medical food could also contain as ingredients other components, comprising e.g. combinations of vitamins, mineral salts and other substances directed at diet supplementing.

One or more of the fractions of the invention or the composition of the invention will therefore be useful for their barrier effect properties and, when present, their mucoadhesion properties, in all those cases in which the protection of skin or mucous membranes is needed or desirable, those may be cases in which a drug that attacks mucous membranes or skin has to be administered, therefore in order to prevent or limit the damaging action of the drug, or in those cases in which the protection of a partially compromised skin or mucous membrane is preferable or desirable so as to enable a better and quicker healing thereof, defending it from further aggressions, or in those cases in which an individual has a chronic disorder in which skin or mucous membranes sustain irritations or alterations, therefore a barrier effect can prevent or limit damages on the skin or the mucous membrane.

The invention also relates to a method for the treatment or for the prevention of the onset or the worsening of skin lesions not involving the presence of open wounds, or for the treatment of open wounds, wherein such method comprises one or more applications of the composition of the invention or of the medical device comprising it once or more per day on the concerned part.

The application of the composition, for instance, could be repeated whenever needed (e.g. at each change of pad in case of prevention of incontinence-related rashes) or once, twice, thrice, four or more times per day in general.

The invention also relates to a method for the preparation of a composition according to any one of the embodiments described above wherein fractions of extract of Helichrysum depleted in active principles and enriched in components giving mucoadhesive and barrier effect properties as described herein (fractions B, D) or mixtures thereof are mixed with at least one of excipients and/or substances of natural and/or plant origin of plants different from Helichrysum having emollient, digestive, pro-kinetics, cholagogue, carminative, prebiotic, relaxing, excipient, preservative, humectant properties as described above.

Among these, e.g., could be used one or more of: gentian root extract, boido leaves extract, milk thistle fruits extract, artichoke leaves extract. dandelion root extract, anise fruit extract, rosemary leaf extract, mint leaves extract, marjoram leaves extract, cumin fruit extract, coriander fruit extract, ginger root extract, fennel fruit extract, caraway fruit extract, chamomile flower extract, althea root extract, linseed extract, barleycorn extract, charcoal, inulin.

Object of the present invention is also a method for the protective (preventive or curative) treatment of the skin or mucous membranes providing the administration of one or more of the fractions of the invention or of the composition of the present invention to a patient in need thereof. Such administration could also be concomitantly with the administration of other drugs.

The reduction of pharmacologically active principles and the increase in mucoadhesive and barrier properties in the fractions of the invention or in the composition makes such products particularly suitable to administration concomitantly with other drugs, as a side effect of interaction between the extract or the composition of the invention and the drug coadministered or concomitantly administered is markedly unlikely.

A non-limiting example of the method of treatment and/or of prevention of the skin or mucous membranes could comprise the administration of a daily dosage, subdivided into a single dose or plural doses, described of the mixture or the composition according to the present description, for a period of time of between one and six weeks, e.g. of between three and six weeks or even fora period of time higher than six weeks, in accordance with the judgment of the treating doctor.

Such administration could precede the administration of the drug even fora prolonged period, so as to optimize the health state of the skin or of the mucous membrane to be treated.

The treating doctor will know how to establish both the most appropriate dosage and the administration times also on the basis of the patient's health state, weight, sex and age.

One of the substantial differences between the structure of the skin and that of the mucous membranes is represented by the absence, in the mucous membranes, of a selective barrier such as the corneous layer. Oral mucous membranes contact with noxious or irritating substances present in the environment (pollutants, pathogenic microorganisms, etc.) can therefore cause a high penetration of said substances both inside the mucous membranes and in the related airways (bronchi, lungs, etc.) causing inflammatory and/or allergic pathologies.

The protective action of the fractions of extract of the present invention was assessed by mucoadhesion assays and barrier effect assays.

The former aim at evaluating the mucoadhesivity of the product under examination in order to establish whether such product has the ability to adhere to mucous membranes, and consequently exert a protective action.

The cytoadhesion assay performed with the fractions of the invention aims at evaluating the mucoadhesivity of the product under examination in order to establish whether such product has the ability to adhere to mucous membranes, and consequently to exert a protective action.

Assessment of the Mucoadhesive Ability of the Extract of the Invention to Mucosal Cells, by Evaluation of the Percentage of Inhibition of the Lectin/Glycoprotein Bond

Mucoadhesivity is determined by evaluation of the percentage of inhibition of the lectin/glycoprotein bond. In this model, for instance, buccal, gastric, intestinal or vaginal cells may be used. Cells were initially treated with biotinylated lectin (Con-A), having high affinity for the glucoside and mannoside residues present in membrane glycoproteins. The sites of the glycoproteins of the mucosal membranes will thus be occupied with the biotinylated lectin. The presence of biotin (vitamin H) in the lectin is indispensable for the next stage. The cells already treated with biotinylated lectin are charged with streptavidin peroxidase, making it possible, thanks to the high affinity between biotin and streptavidin, to form the protein-glucose-lectin-biotin-streptavidin peroxidase complex. At this point, the cells are washed and the protein-glucose-lectin-biotin-streptavidin peroxidase complex is quantified, thanks to the presence of peroxidase, by means of a reaction of oxidation of the ortho-phenylenediamine.

The protein/glucose/lectin/biotin/streptavidin peroxidase complex catalyses the polymerization reaction:

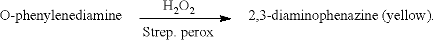

The intensity of the yellow/orange coloration of the solution (measured using a spectrophotometer with =450 nm) is proportional to the quantity of glycoprotein/lectin bonds and therefore to the quantity of available sites (glycoproteins) for mucoadhesion. The absorbance value thus determined constitutes the “control”.

In the mucoadhesion assay reported below, the gastrointestinal cells are treated for about 15 minutes at 30° C. with the product under examination before the treatment with lectin. In the presence of mucoadhesive products, said products will inhibit lectin bonding, decreasing, proportionally to their mucoadhesion ability, the signal strength in the sample compared to the control as described above.

The percentage of mucoadhesion of the product (% MA) could be determined as

% MA=(1−abs sample/abs control)×100

Barrier Effect Assay

The barrier effect assay is a biological-type in vitro assay employed for assessing the ability of finished products and/or raw materials to protect, through the formation of a thin “insulating” layer, mucous membranes and skin from contact with environmental contaminants (dust, pollens, microorganisms, etc.)

The assay was developed to simulate in vitro the action exerted by products that are applied on skin and/or mucous membranes with the aim of creating a protective film against external aggressors.

The model takes advantage of the principle whereby cells subjected to contact with an inflammatory agent produce and secrete pro-inflammatory mediators (cytokines) in the extracellular environment in an amount related to the degree of inflammation caused; within a certain range, a direct proportionality exists between concentration and times of exposure to the inflammatory agent and amounts of cytokines released.

The experimental model adopted provides two chambers physically separated by a semi-permeable membrane (0.4 μm pores). Cells are seeded in the lower chamber, whereas the upper chamber accommodates the inflammatory agent; on the semi-permeable membrane separating the two chambers a thin film of the sample under analysis is stratified to highlight, if present, a barrier effect to the free transit of the inflammatory agent.

The semi-permeable membrane allows passage of the inflammatory agent in the lower chamber and constitutes the support on which the sample to be assayed is stratified. Depending on the “insulating” ability of the sample, a decrease of LPS migration from the upper chamber to the bottom one will be had (therefore a lesser stimulation of cells to cytokine production).

### EXAMPLES

### Example 1

Preparation of Fractions B and D

There were performed two steps of extraction from Helichrysum tops in ethanol at decreasing concentrations, performing a step in 96% ethanol, and a step in 5% ethanol.

The alcoholic extracts obtained as described were gathered in a 50:50 ratio and subjected to centrifugation for a time of 1-5 minutes at a rotation rate equal to 3,500 rpm; a precipitate and a supernatant constituting a clarified alcoholic fraction were obtained. The clarified alcoholic fraction was concentrated by ethanol evaporation with use of a thin film distillation system according to the standard protocol, providing the feeding of the extract to be concentrated at a rate of about 500 1/h; operation was by setting a residual vacuum of 0.6-0.8 bars and the fluid heating the evaporation walls was set at 140° C., thereby obtaining, after ethanol elimination, a concentrated aqueous fraction (fraction B) and a precipitate. Fraction B has a high mucoadhesive power, equal to a value of 68% when measured by mucoadhesion assays on cells, and a barrier effect measured by the assay described herein equal to about 49%.

Fraction B was filtered on a panel filter with a cutoff of 25 micrometers. The filtrate thus obtained was subjected to a step of ultrafiltration/nanofiltration on membrane with a cutoff of 2500 daltons and/

The filtrate of the ultrafiltration/nanofiltration (Fraction D) thereby obtained has a high mucoadhesive power equal to a value of 74% when measured by mucoadhesion assays on cells and a barrier effect measured by the assay described herein equal to that of the starting extract.

### Example 2

Measurement of Total Flavonoids in the Fractions

Determination of total flavonoids expressed as isoquercitrin. Sample extraction:

0.30g of sample were accurately weighed in a 100 ml flask. Adding 1 ml of hexamethylenetetramine (5 g/l), 50 ml of acetone and 2 ml of diluted HCL (70 g in 100 ml water), and they were heated 30 minutes under reflux. The same extraction was repeated other two times on the residue of the first extraction, and the three extracts were gathered together in an 100 ml volumetric flask.

20 ml of the solution so obtained were obtained into an separating funnel, 20 ml of ultrapure water were added, and subsequently an extraction was carried out, first with 15 ml and then for other three times with 10 ml of ethyl acetate. After each extraction, phases were separated and the organic phase was recovered in a 100 ml Erlenmeyer flask. The gathered organic extracts were introduced into a second separating funnel. Other two extractions were performed, with successive portions (of 50 ml each) of ultrapure water.

After each extraction the phases were separated, filtered with cotton and with anhydrous sodium sulfate, and the extracts were decanted into a 50 ml flask and then brought to volume with ethyl acetate.

Assay solution: 10 ml of the solution obtained as described above were put into a 25 ml volumetric flask. 1 ml of aluminum chloride in acetic acid solution in methanol (2% AlCl₃ in acetic acid solution in 5% v/v methanol) was added, and it was brought to volume with the acetic acid solution in 5% (v/v) methanol.

Reference solution. 10 ml of this solution were put into a second 25 ml volumetric flask, and the whole was brought to volume with a solution of acetic acid in 5% v/v methanol without addition of AlCl₃.

Spectrophotometric Reading:

After 30 minutes of reaction with AlCl₃ the absorbance of the assay solution was measured at 425nm, using as blank the reference solution.

Calculations:

The percentage content of total flavonoids was calculated on the basis of the molar extinction factor of isoquercitrin with the following formula:

% =(A*1.25)/p

Where A=absorbance measured at 425nm in the assay solution
P=sample weight, expressed in grams.

### Example 3

Assessment of the mucoadhesive ability of the fractions B and D of the invention to mucosal cells, by evaluation of the percentage of inhibition of the lectin/glycoprotein bond

Mucoadhesivity was determined by evaluation of the inhibition percentage of the lectin/glycoprotein bond on intestinal mucosal cells.

2 ml cell suspensions containing 1 million of intestinal mucosal cells (HT-29 ATCC® HTB-38™) were prepared, which were then pretreated with 5 ml of fractions B or D as described herein and analogous samples with non-pretreated cells which were used as control. Cells were left to incubate for 15 minutes under slow stirring at 30° C. in a thermostated bath. Then, they were centrifuged 5 minutes at 2000rpm, supernatant was discarded, and they were washed thrice with TBS (Tris buffered saline) buffer.

The cells (sample and control) were then treated with 5 ml of biotinylated lectin (Con-A) 10 mg/1 for 30 minutes at 30° C., under slow stirring. Then, they were centrifuged 5 minutes at 2000 rpm, the supernatant was discarded and they were washed 3 times with TBS (Tris buffered saline) buffer. The cells already treated with biotinylated lectin were charged with streptavidin peroxidase (5 ml of streptavidin peroxidase, 5 mg/L) for 60 minutes at 30° C. under slow stirring. Then, they were centrifuged 5 minutes at 2000rpm, the supernatant was discarded and they were washed 3 times with TBS (Tris buffered saline) buffer.

To each sample (250,000 cells for each sample) 2.5 ml of O-phenylenediamine (o-pd) in 0.05 M phosphate citrate and H₂O₂ were added, to a final concentration equal to 0.04%. After 2 minutes the reaction was stopped with 1N H₂SO₄. The protein-glucose-lectin-biotin-streptavidin peroxidase complex was subsequently quantified by spectrophotometric reading (X=450 nm), thanks to the presence of peroxidase, by means of a reaction of oxidation of the ortho-phenylenediamine.

The protein/glucose/lectin/biotin/streptavidin peroxidase complex catalyses the polymerization reaction:

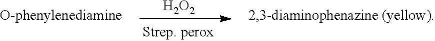

The intensity of the yellow/orange coloration of the solution is proportional to the quantity of glycoprotein/lectin bonds and therefore to the quantity of available sites (glycoproteins) for mucoadhesion. The absorbance value thus determined constitutes the “control”. As explained above, in the presence of mucoadhesive products, an inhibition of lectin bonding to mucosal cells is had which leads to a decrease, proportionally to the mucoadhesion ability of the analyzed sample, of the signal strength in the sample compared to the control as described above. The mucoadhesive ability is expressed as percentage of inhibition of the glycoprotein/lectin bonding, or better as percentage of mucoadhesion of the product according to the equation:

% MA=(1−abs sample/abs control)×100

### Example 4

Barrier effect assay performed for each of the fractions B and D and for the reference extract

(For the barrier effect assay, two chambers physically separated by a semi-permeable membrane (0.4 μm pores) were used. Human fibroblast cells were seeded in the lower chamber, whereas the inflammatory agent LPS (purified E. Coli lipopolysaccharide) was introduced into the upper chamber; on the semi-permeable membrane separating the two chambers a thin film of fractions of extract as described in the present invention was stratified.

Induced inflammatory response was estimated through semi-quantitative dosage of Interleukin 6 (IL6) cytokine released in the culture medium of the lower chamber: barrier effect assessment was obtained by comparison with the positive control in which the two chambers were separated by the same type of semi-permeable membrane, free however from any barrier.

The greater the barrier effect exerted by the stratified film of substance, the lesser the inflammatory action observed in the cells present in the chamber therebelow, as the inflammatory agent in the chamber thereabove will have greater difficulty to cross the semi-permeable chamber.

As to the threshold value above which it may be said that a substance causes a barrier effect in the assay reported herein, a value equal to 15% of inhibition as compared to the control was identified by the Inventors, during the setting up of the assay, on the basis of assays carried out on substances known to have a barrier effect.

Then the barrier effect of the fractions of extract as described herein was evaluated.

The data reported below show how the fractions of extract according to the description have a remarkable barrier effect, compared to the reference extract.

The assessment assay was carried out as follows through a method developed to simulate in vitro the protective action of substances and formulations which, when applied to the skin and mucous membranes in vivo, form an “insulating” film against environmental agents.

The model takes advantage of the principle whereby cells subjected to contact with an inflammatory agent produce and secrete pro-inflammatory mediators (cytokines) in the extracellular environment in an amount related to the degree of inflammation caused. The greater the amount of inflammatory agent reaching the cells, the greater the amount of cytokines released.

The model envisages the arrangement of two chambers physically separated by a semi-permeable membrane which allows the passage of sufficiently small-sized solutes.

In the lower chamber, consisting of a well containing plates for cell cultures, HuDe cells (no. BS PRC 41, purchased from the Istituto Zooprofilattico di Brescia, Italy) are grown, while the upper chamber, consisting of an insert for complex cell cultures (transwells), accommodates the inflammatory agent. On the surface of the semi-permeable membrane of the insert separating the two chambers, before the introduction of the inflammatory agent in the upper chamber, a thin film of the sample being examined is stratified to assess any BE upon the free passage of the inflammatory agent.

As a function of the insulating capabilities of the sample, there will be had a decrease of the migration of the inflammatory agent from the upper chamber and, as a consequence, a lesser stimulation of the cells to produce cytokines. The extent of the inflammatory reaction is estimated through the semi-quantitative dosage of cytokines released in the culture medium of the lower chamber, in particular of interleukin 6 (IL-6).

As a control a similar experiment is used in which no sample is stratified on the membrane, thus making it possible to measure the effect of the inflammatory agent without any barrier in addition to the semi-permeable membrane.

Further, an internal control is used in which the cultured cells are pre-treated with the substance adapted to induce the release of the marker and the sample is placed on the semi-permeable membrane in the absence of said substance, one or more measurements in time are thus performed of the quantity of marker in the culture medium of said internal control. In the internal control, the cells are therefore stimulated first with the inducing substance and then there has to be assessed whether the sample which may pass the membrane and go into the cells pushed by the medium above has any effect in decreasing the release of the marker not related to the barrier effect. For instance, when an inflammatory agent is used as the inducing substance, the internal control makes it possible to understand if the reduction in the concentration of cytokines in the culture medium is due to the barrier effect or if the sample that may pass into the cells pushed by the medium above has any effect in reducing the inflammatory response independently of the barrier effect.

The barrier effect (BE) is expressed as a percentage of the reduction of the release of IL-6 and is calculated through comparison with the positive control in which the two chambers are separated by the same type of semi-permeable membrane without the barrier created by the sample.

4.1 Preparation of the Cell CUlture:

For each assayed sample, HuDe line cells were seeded in the wells of a cell culture plate, one for the barrier assay (BA) and another one for the internal control at a density of 40,000 cells/ml in MEM medium supplemented with 10% bovine serum (FBS); 1 ml of cell suspension per well.

The cells were treated with the SAMPLE (CAM), with the POSITIVE CONTROL (C+) (inflammatory agent without sample), and with the NEGATIVE CONTROL (C-) (medium only) and each assay was carried out in triplicate. The plates were incubated at 37° C., overnight (22-24 hours).

4.2 Preparation of Insserts for Complext Cell Cultures

Cell Culture Inserts (Becton Dickinson) for complex cell cultures were placed on other plates, and on each of them a fixed amount of collagen of 0.1 mg/ml was supplied. The plates were incubated at 37° C., overnight (22-24 hours). 4.3 VERIFICATION OF STATE AND LEVEL OF CELL CONFLUENCY In order to proceed with the experiment, a confluency not lower than 95% is required.

4.4 Collagen Layer Drying

From the two plates (BA and IC) with the inserts the collagen was removed and the insert left under the flow of the hood for the time required to let them dry completely (10+15 minutes).

4.5 Barrier Assays (BA)

The steps described below were carried out in the culture plate for the BA.

### Setting up on the Sample Layer in the BA:

On the sample's semi-permeable membrane 100 μl of a composition based on each of the fractions of extract (2%) (the assay was performed for each fraction) described in the invention were inoculated and allowed to stratify for 20 minutes, while in the C+ and C− inserts nothing was added. Once the 20 minutes had elapsed, excess sample was eliminated and the membranes washed with PBS according to procedures specified by the protocol.

Addition of LPS (Inflammatory Agent) to BA Inserts

Once the sample layer had dried, in the first three CAM inserts and in the three C+ones, 300 μl of the LPS (membrane lipopolysaccharide) solution were inoculated at the concentration of 1 μg/ml while in the remaining three of the C− 300 μl of MEM medium with 5% FBS were added. The inserts were inserted in their respective wells with the cells and the plates were incubated for 1 h at 37° C. and under an atmosphere enriched with 5% CO₂ overnight (22-24 hours).

Once completed the 1 h incubation, the inserts were removed and discarded and the plates were incubated again overnight (22-24 hours).

4.6 Internal Control (OC) Assay:

The internal control assay was carried out concomitantly with the BA. Exposure of IC cells to LPS:

Once dried, in the first six inserts of IC, three for the sample to be analyzed, CAM, and three for the C+, 300 μl of the LPS solution were inoculated, while in the remaining three of the C− 300 μl of the medium were added.

The inserts with LPS and MEM were then inserted in the wells with IC cells and all incubated for 1 h.

LPS Removal and IC Membrane Drying:

Once completed the 1 h incubation, the inserts were removed from the wells with the cells and transferred to the empty plate, while the plate with the cells was placed in an incubator.

The LPS solution still present was removed from the inserts, the latter were subjected to a rapid wash with ultrapure sterile water and allowed to dry.

Arrangement of sample layer in the IC:

On the semi-permeable membrane of the three inserts for the sample, 100 μl of a composition based on each of the fractions of extract (2%) according to the invention were inoculated and allowed to stratify for 20 minutes while in the C+ and C− inserts nothing was added. Once the 20 minutes had elapsed, the excess sample was eliminated and the membranes were washed with PBS according to procedures specified by the protocol.

Addition of LPS to IC Inserts

Once the inserts with the sample were ready, 300 μl of medium were added to all inserts (CAM, C+, C−). The inserts were inserted in their respective wells with the cells and the plates incubated for 1 h at 37° C.

Once completed the 1 h incubation, the inserts were removed and discarded and the plates incubated again overnight (22-24 hours).

4.7 Supernatant Collection and Enzyme Immunoassay

Once the 22-24 hours had elapsed, the supernatants were collected from the BA and the IC plates for performing the ELISA assay and the semi-quantitative dosage of IL-6.

### Barrier Effect (BE) Assessment

The BE of a substance or compound is expressed as % reduction in the release of IL-6 cytokine by cells exposed to LPS wherein the sample has been assayed relative to the positive control (C+) wherein the cells have only been exposed to LPS.

BE=% reduction in release of IL-6 cytokine=100−[(pg/μL cytokines released from sample/pg/μL cytokines released from C+)×100]

Table 1 reported above shows that the initial extract of Helichrysum examined does not have a detectable mucoadhesive effect, whereas fractions B and D have a mucoadhesive effect much superior to that of the starting extract (BE extract non measurable, fractions B and D of said BE extract measurable) associated with a keeping or even with an increase of barrier effect properties of the fractions with respect of the starting extract.

The data obtained show that the fractions of the invention are suitable for the uses described and claimed.

1. A fraction of an extract of Helichrysum characterized by a mucoadhesion activity superior to that of said extract and a barrier effect greater than or equal to that of said extract.

2. The fraction of extract of Helichrysum according to claim 1 wherein said mucoadhesion activity is measured as a mucoadhesion percentage in a mucoadhesion assay on cells.

3. The fraction of extract of Helichrysum according to claim 3 wherein said mucoadhesion percentage is greater than 60%, or greater than 65% or greater than 70%.

4. The fraction of extract of Helichrysum according to claim 1 wherein said fraction is also characterized by a barrier effect greater than or equal to that of said extract.

5. The fraction of extract of Helichrysum according to claim 4 wherein said fraction is characterized by a barrier effect percentage measured by cellular markers release inhibition assays in response to an inflammatory or irritating agent greater than or equal to 30%, or greater than or equal to 32%.

6. The fraction of extract of Helichrysum according to claim 5 wherein said fraction is characterized by a mucoadhesion percentage measured by mucoadhesion assay on cells greater than 65% and a barrier effect percentage measured by cellular markers release inhibition assays in response to an inflammatory or irritating agent greater than 45%.

7. The fraction of extract of Helichrysum according to claim 5 wherein said fraction is characterized by a mucoadhesion percentage measured by mucoadhesion assay on cells greater than 70% and a barrier effect percentage measured by cellular markers release inhibition assays in response to an inflammatory or irritating agent greater than or equal to 30% or greater than or equal to 32%.

8. The fraction of extract of Helichrysum according to claim 1 wherein said extract is an extract of Helichrysum flowering tops optionally in a dried or lyophilized form.

9. The fraction of extract of Helichrysum according to claim 8 wherein said extract is a hydroalcoholic extract.

10. A composition comprising one or more fractions of extract of Helichrysum according to claim 1 and not comprising a non-fractioned extract of Helichrysum.

11. A carrier for pharmaceutical or herbal compositions comprising one or more fractions of extract of Helichrysum according to claim 1, characterized by having a mucoadhesion percentage measured by mucoadhesion assay on cells greater than 60%, greater than 65% or greater than 70% and a barrier effect percentage measured by cellular markers release inhibition assays in response to an inflammatory or irritating agent, greater than or equal to 30% or greater than or equal to 32%.

12. A process for the preparation of fractions of extract of Helichrysum comprising:
a. preparing a hydroalcoholic extract of Helichrysum flowering tops and subjecting said extract to centrifugation and/or decantation, thereby obtaining a precipitated fraction and a clarified alcoholic fraction;
b. obtaining a concentrated aqueous fraction B from the clarified alcoholic fraction, wherein said fraction B is characterized by a mucoadhesion activity superior to that of said extract and by a barrier effect greater than that of said extract.

13. A process according to claim 12 wherein said fraction B is characterized by a percentage of mucoadhesion measured by mucoadhesion assay on cells greater than 65% and a percentage of barrier measured by cellular markers release inhibition assays in response to an inflammatory or irritating agent greater than 45%.

14. A process according to claim 12 further comprising:
c. decanting and/or centrifuging and filtering said fraction B, thereby collecting a water-soluble fraction;
d. subjecting said water-soluble fraction to member ultrafiltration or nanofiltration and collecting a fraction D, corresponding to the permeate obtained from said ultrafiltration or nanofiltration, wherein said fraction D is characterized by a mucoadhesion activity superior to that of said extract and by a barrier effect greater than or equal to that of said extract.

15. The process according to claim 14 wherein said fraction D is characterized by a mucoadhesion percentage measured by mucoadhesion assay on cells greater than 70% and a barrier effect percentage measured by cellular markers release inhibition assays in response to an inflammatory or irritating agent greater than or equal to 30% greater than or equal to 32%.

16. A method for protective treatment of skin or mucous membrane of a patient in need therefore, the method comprising administering (i) one or more fraction(s) of an extract of Helichrysum characterized by a mucoadhesion activity superior to that of said extract and a barrier effect greater than or equal to that of said extract or (ii) a composition comprised of said fraction(s) and not comprised of a non-fractioned extract of Helichrysum to said patient.
